(19) United States
(12) Patent Application Publication
(10) Pub. No.: US 2025/0018172 A1
(43) Pub. Date: Jan. 16, 2025

(54) CONVERTER FOR POWER SUPPLY OF MEDICAL DEVICES

(71) Applicant: Elias SIORES, Athens (GR)

(72) Inventor: Elias SIORES, Athens (GR)

(21) Appl. No.: 18/718,995

(22) PCT Filed: Dec. 14, 2022
(86) PCT No.: PCT/GR2022/000071

§ 371 Date: Jun. 12, 2024

(30) Foreign Application Priority Data
Dec. 14, 2021 (GR) 20210100876

(51) Int. Cl. A61M 60/882; A61M 60/178; A61M 60/216; A61M 60/403; A61N 1/378; A61N 1/39

(52) U.S. Cl. CPC A61M 60/882; A61M 60/178; A61M 60/216; A61M 60/403; A61N 1/3785; A61N 1/3975

(57) ABSTRACT

A convertor (100) for converting the mechanical energy of the movement of the heart muscle, or of other moving organs, into electrical energy comprising two flexible blocks (7, 7′), wherein a first and a second end of a plurality of extendable elements (1) are immovably connected on the first and on the second flexible blocks (7, 7′). Each of the extendable elements (1) comprises a layer of triboelectric material and a hollow portion (6). Each of the plurality of the extendable elements (1) comprises a triboelectric spiral structure (3) that is mounted within the hollow portion (6), wherein the triboelectric spiral structure (3) comprises a plurality of twisted elongated elements (4) that are movable within the hollow portion (6) of the extendable elements (1). Each of the twisted elongated elements (4) has the shape of a rod and each of the twisted elongated elements (4) comprises a triboelectric material layer (2) and a conducting material layer (12).

This application claims priority from the Greek patent application GR20210100876, filed on Dec. 14, 2021, the entire content of which is incorporated herein by reference.

### FIELD

The present disclosure relates to the field of convertors used for the power supply of medical devices that are mounted in the human body.

The present disclosure generally pertains to a power source whose energy is derived from changes in shape responsive to autonomic movements of the human body. More, the disclosure refers specifically to an implantable convertor constructed to be a power source, such that movement of the heart and thoracic cavity acting on the power source will cause the generation of electrical power.

### BACKGROUND

Common medical devices such as heart pacemakers, defibrillators, Left Ventricle Assist Devices (herein after called LVADs), and neuro-stimulators are traditionally used either to continuously monitor a patient's cardiac rhythm and function and deliver, if necessary, to the heart electrical pulses in case a rhythm disorder is detected by the device, or to help pump blood from the heart to the body as in the case of the LVADs. Such medical devices consume significantly large amounts of energy for their proper function. Currently, their function is based either on electrical energy storage devices which are implanted in the human body, for example with the heart pacemaker or the defibrillator or to external portable energy accumulators that communicate with, for example, the LVADs, through the human skin. These appliances show however several drawbacks such as the need for battery replacement of devices, such as the heart pacemaker, after some years of continuous operation which, inter alia, requires a short surgical operation for the patient. Such procedures increase the risk of infection and other complications, which not rarely lead to severe morbidity, or mortality, and pose a significant clinical and economic burden. In addition, another disadvantage is that, for example for the LVADs, transportation of portable bulky energy storage devices is required to ensure proper functionality of the medical device. Similar drawbacks appear also to other implantable medical devices that have as a basis for their functionality the electrical energy.

A solution to increase the lifetime of such a medical device (a pacemaker, a defibrillator) is by efficiently providing auxiliary power to the power-consuming device. This could be possible through an implanted assembly, capable of harvesting the energy produced by the mechanical work done by a human's body, which would be otherwise dissipated, and converting it to electrical power. An appealing internal power source generator is the heart-thoracic cavity system due to its ability to continuously deliver mechanical energy as long as the human is alive. So, one could exploit the considerable energy produced therein, by placing a mechanical-to-electrical power transformer onto the heart itself or the diaphragm and provide electrical power to any medical device that uses electrical power.

There is thus the need to provide an implantable that will overcome the previously mentioned drawbacks of the medical devices by transforming the mechanical energy produced by a contacting applied force, such as that produced by the autonomic movement of a mass of a patient's organ, as is the hearts or the diaphragm's mechanical contraction/expansion into electrical energy. Of course, it is contemplated that inside the thoracic cavity, several movements take place, such as by heart movement, lung expansion, and diaphragm movement and serve as global energy harvesting sources. The electrical energy produced by such movements is then delivered to a power management and storage module capable of powering the medical device (pacemaker, or other selected device) with energy.

EP2975759A1 discloses Triboelectric Nanogenerators and materials that take advantage of the triboelectric phenomena to produce electrical energy. The triboelectric nanogenerator is comprised of two friction material layers coming in contact periodically only on one of their surfaces, which are inherently connected to two respective conductive material layers, on their non-contacting surface. In earlier in-vivo energy harvesting attempts, the piezoelectric phenomenon was exploited in response to the heart movement, in order to provide a supply for pacemakers and other implantable devices. US2010160994A1 discloses a power source whose energy is derived from changes in shape responsive to autonomic movements of the human body. There is described a self-contained power source configured to be implanted in a human's heart, such that movement of the heart acting on the power source will cause the generation of electrical power. In an example, there is disclosed an implant comprising a power-consuming means for responding to a physiological requirement of the body and a power source having a sheathed flexible piezoelectric assembly configured to generate an electrical current when flexed by tissue in the human body, wherein the piezoelectric assembly comprises a plurality of oriented nanowires arranged in an array and forming a nanowire layer, and wherein the plurality of oriented nanowires is encapsulated in a polymeric matrix.

### SUMMARY

The present disclosure addresses the widely recognized need to eliminate surgical procedures for battery replacement in medical devices, which is known to cause problems to patients and at the same time provide patients with a lifetime source of energy feeding the pacemaker or other selected device.

The current disclosure introduces an convertor comprising a power harvesting architecture utilizing triboelectric phenomena and providing sufficient power generation to supply medical devices during the patient's lifetime without the need of changing batteries. The construction is deployable at a heart, preferably in its entirety, or at other moving organs, such as the lungs and the diaphragm. The electrical system includes a triboelectric nanogenerator, a voltage stabilizer-energy management device, and an accumulator. The triboelectric nanogenerator comprises a plurality of extendable elements which comprise a plurality of triboelectric spiral structures twisted together inside a hollow portion of the elements, for apposition to the heart muscle for generating electric power from the heart muscle-thoracic cavity movement. The triboelectric nanogenerator is configured to be implanted within a patient at a location that subjects the triboelectric nanogenerator to periodic movement. Examples of periodic movement include breathing and the movement caused by a beating heart. Another example includes skeletal muscles and the body movement that can be generated via the skeletal muscles.

According to aspects of the present disclosure, an convertor implantable inside a human body is provided that includes a power-consuming means responsive to the physiological or pathological requirement of the human body, a power source, and a power storage device. The power source comprises a sheathed triboelectric nanogenerator assembly that is configured to generate an electrical current when flexed by the tissue of the body and communicate the generated current to a power storage device which is coupled to the power consuming means. The present disclosure, therefore, pertains to a power source whose energy is derived from changes in shape responsive to autonomic movements of the human body.

The advantage of the present disclosure over known convertor, such as the ones based on piezoelectric elements is that it best exploits the triboelectric phenomena, optimized for the in-vivo environment, especially due to the low frequency of the human movements (below 4 Hz), in order to sustainably produce more energy. Moreover, it is architecturally designed to maximize the energy harvesting potential.

According to a first aspect, an implantable convertor is provided for converting the mechanical energy caused by the movement of the heart muscle during the systole-diastole cycle and the negative and positive forces applied to the inspiration into electrical energy, the convertor comprising a first flexible block, a second flexible block, wherein a first and a second end of a plurality of extendable elements are inherently immovably connected on the first and on the second flexible block, wherein each of the plurality of extendable elements comprises a layer of triboelectric material and a hollow portion, wherein each of the plurality of the extendable elements comprises a triboelectric spiral structure that is mounted within the hollow portion, wherein the triboelectric spiral structure comprises a plurality of twisted elongated elements that are movable within the hollow portion. The hollow portion therefore is harbouring a triboelectric spiral structure, in an example of three elongated elements, that are twisted together along the longitudinal axis of each extendable element within the hollow portion, that are movable within the hollow portion of each extendable element. In examples, both extendable elements and the twisted elongated elements comprise a distinct set of triboelectric and conducting material layers, where the triboelectric layers of the extendable elements and the elongated elements come in direct contact by movement, while their respective electrical conducting material layers do not contact each other.

In embodiments, each of the plurality of the elongated elements has the shape of a rod and wherein each of the elongated elements comprises a triboelectric layer and a conducting layer.

In embodiments, the triboelectric layer of the twisted elements and the triboelectric layer of the elongated elements comes in direct contact by movement.

In embodiments the first and the second flexible blocks are of parallelepiped or cylindrical shape.

In embodiments, the plurality of rods consists of three rods.

In embodiments, the length of the first flexible block may be about 0,5-0,7 of the length of the second flexible block, where to be implanted on the heart.

In embodiments, the length of the first flexible block is 0,6 of the length of the second flexible block, where to be implanted on the heart.

In embodiments, each of the plurality of extendable elements has a cylindrical shape.

In embodiments, the plurality of extendable elements is made of a flexible material having an original length, said flexible material is capable of elongating by about 25-40% compared to its original length and configured to return to its original length, upon ending its elongation.

In embodiments, the first and second flexible block each comprising a pair of ends, wherein ends of the first flexible block are connected to form a circle.

In embodiments, the convertor is coated with a mixture of an electrically insulating material and an anti-fibrotic solute.

In embodiments, the anti-fibrotic solute may comprise magnesium salt and dimethoxyphenyl-propanol-aminobenzoic acid.

In embodiments, the first and second flexible blocks may further comprise a first end and a second end and a through hole extending longitudinally from the first end to the second end of each of the first and second flexible blocks.

In embodiments, each elongated element comprises a electrical connection that is wrapped under tension to the elongated element.

In embodiments, each conducting material layer of each elongated element from the plurality of elongated elements may be coupled with a metal wire, said wire extending through the through hole of each flexible block.

In embodiments, each conducting material layer of each extendable element, from the plurality of extendable elements, may be a physical continuum of the same material of the flexible blocks.

In embodiments, use of the convertor is disclosed with a system comprising a voltage stabilizer-energy management device and an accumulator, configured to produce and store electrical power derived by the contraction and the expansion of the heart of a human body.

### BRIEF DESCRIPTION OF THE DRAWINGS

FIG. 1 is a 2-D schematic illustration of a longitudinal-section of the front view of an implantable convertor harvesting natural power from the heart-thoracic cavity movement.

FIG. 2 is a cross-sectional view of an extendable element comprising a plurality of rods.

FIG. 3 is a longitudinal cross-sectional view of an extendable element according to the present disclosure.

FIG. 4a is a partial detailed schematic illustration of the convertor of the present disclosure showing the second flexible block.

FIG. 4b is a partial detailed schematic illustration of the convertor of the present disclosure showing the first flexible block.

FIG. 5 is a partial detailed illustration of an embodiment of the present disclosure showing the metal wire and the electrical connection.

FIG. 6 is a partial detailed illustration of the second flexible block without the external insulating material, with the longitudinal through hole.

FIG. 7 shows an embodiment of the present disclosure where the convertor is mounted on a heart muscle.

### DETAILED DESCRIPTION

It is commonly known in the art that for the treatment of a plurality of pathological conditions of humans, medical electronic devices are used such as but not limited to, heart pacemakers, defibrillators, LVADs, and neurostimulators. Such devices have the drawback of consuming variable and often large amounts of energy for their proper function. Further, such medical devices require the provision of either integrated energy accumulators, for example in pacemakers and in defibrillators, or portable external accumulators that communicate through the skin with the main device, for example in LVADs. It is, therefore, necessary to proceed with the replacement of the battery of such medical devices on a frequent basis, to ensure that they operate properly and to avoid putting at risk the health of a patient. In addition, as in the case of LVADs, existing batteries are not adequate to power their function, and the external accumulators are often of significant volume, thus causing inconvenience to the patient when they need to move or carry out any everyday activity and provide a source of infection. This technological weakness is the main restriction, of a greatly needed expansion of their clinical implementation in broader patient populations.

It is thus an object of the current disclosure to overcome the aforementioned drawbacks and provide an convertor that in combination with a current stabilizer-energy management device and an accumulator located within the human body will provide continuous and reliable operation of such medical devices, through the conversion of the mechanical energy, for example, of the movement of the heart and the thoracic cavity of a patient to electrical energy. Such convertors are foreseen to be implanted at the same time as the medical device, thus also eliminating the need for any battery replacement that may be required due to the continuous operation of the medical device.

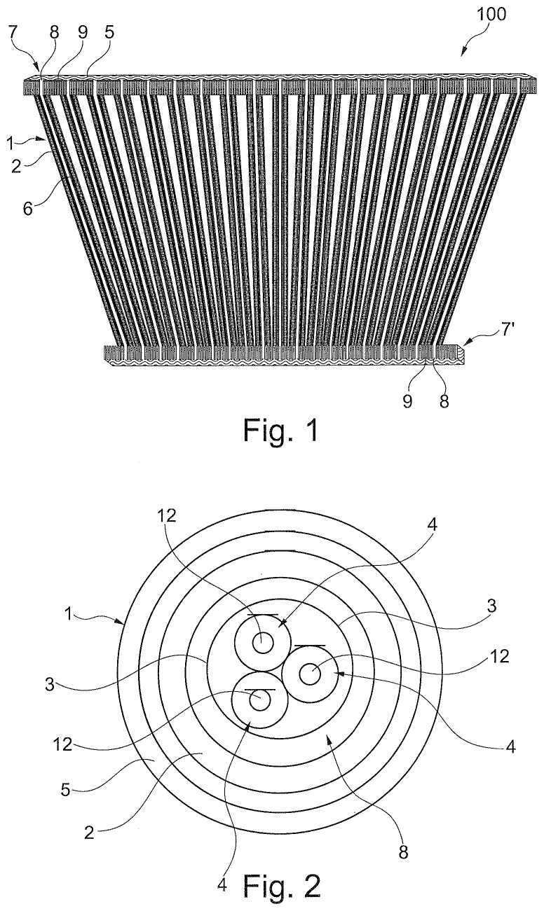

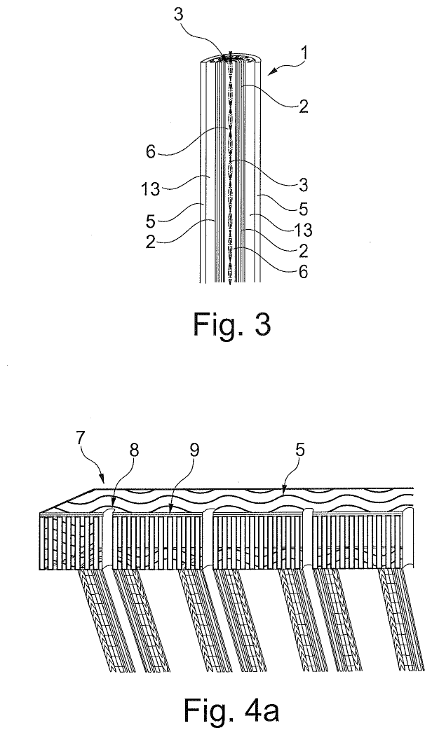

An embodiment of the convertor according to aspects of the disclosure will now be described with reference to FIGS. 1 to 7. Although the convertor is described with reference to specific examples, it should be understood that modifications and changes may be made to these examples without going beyond the general scope as defined by the claims. In particular, individual characteristics of the various embodiments shown and/or mentioned herein may be combined in additional embodiments. Consequently, the description and the drawings should be considered in a sense that is illustrative rather than restrictive. The Figures, which are not necessarily to scale, depict illustrative aspects and are not intended to limit the scope of the disclosure. The illustrative aspects depicted are intended only as exemplary.

The term “exemplary” is used in the sense of “example,” rather than “ideal.” While aspects of the disclosure are amenable to various modifications and alternative forms, specifics thereof have been shown by way of example in the drawings and will be described in detail. It should be understood, however, that the intention is not to limit aspects of the disclosure to the particular embodiment(s) described. On the contrary, the intention of this disclosure is to cover all modifications, equivalents, and alternatives falling within the scope of the disclosure.

Various materials, methods of construction, and methods of fastening will be discussed in the context of the disclosed embodiment(s). Those skilled in the art will recognize known substitutes for the materials, construction methods, and fastening methods, all of which are contemplated as compatible with the disclosed embodiment(s) and are intended to be encompassed by the appended claims.

As used in this disclosure and the appended claims, the singular forms “a,” “an,” and “the” include plural referents unless the content clearly dictates otherwise. As used in this disclosure and the appended claims, the term “or” is generally employed in its sense including “and/or” unless the content clearly dictates otherwise.

Throughout the description, including the claims, the terms “comprising a,” “including a,” and “having a” should be understood as being synonymous with “comprising one or more,” “including one or more,” and “having one or more” unless otherwise stated. In addition, any range set forth in the description, including the claims should be understood as including its end value(s) unless otherwise stated. Specific values for described elements should be understood to be within accepted manufacturing or industry tolerances known to one of skill in the art, and any use of the terms “substantially,” “approximately,” and “generally” should be understood to mean falling within such accepted tolerances.

When an element or feature is referred to herein as being “on,” “engaged to,” “connected to,” or “coupled to” another element or feature, it may be directly on, engaged, connected, or coupled to the other element or feature, or intervening elements or features may be present. In contrast, when an element or feature is referred to as being “directly on,” “directly engaged to,” “directly connected to,” or “directly coupled to” another element or feature, there may be no intervening elements or features present. Other words used to describe the relationship between elements or features should be interpreted in a like fashion (e.g., “between” versus “directly between,” “adjacent” versus “directly adjacent,” etc.).

Spatially relative terms, such as “top,” “bottom,” “middle,” “inner,” “outer,” “beneath,” “below,” “lower,” “above,” “upper,” and the like, may be used herein for ease of description to describe one element or feature's relationship to another element(s) or feature(s) as illustrated in the drawings. Spatially relative terms may be intended to encompass different orientations of a device in use or operation in addition to the orientation depicted in the drawings. For example, if the device in the drawings is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Although the terms “first,” “second,” etc. may be used herein to describe various elements, components, regions, layers, sections, and/or parameters, these elements, components, regions, layers, sections, and/or parameters should not be limited by these terms. These terms are only used to distinguish one element, component, region, layer, or section from another region, layer, or section. Thus, a first element, component, region, layer, or section discussed herein could be termed a second element, component, region, layer, or section without departing from the teachings of the present inventive subject matter.

As shown in FIG. 1, the convertor 100 may include minimal components to simplify manufacturing, such as a first flexible block 7, a second flexible block 7′, wherein the first and second flexible blocks being arranged to operate in substantially parallel plans. Further, the convertor 100 comprises a plurality of extendable elements 1 each having a first end and a second end, said first and second end being inherently immovably connected on the first and second flexible blocks (7, 7′) of the convertor 100. In examples, each of the plurality of extendable elements 1 has a triboelectric material layer 2 and a hollow portion 6 where it encompasses a triboelectric spiral structure 3 that is placed within the hollow portion 6 of each of the plurality of extendable elements 1 of the convertor 100. Such convertor belong to the field of Triboelectric Nanogenerators (TENG) and due to the triboelectric phenomena, they convert the mechanical energy of an applied external force to electrical energy.

The present disclosure can be implemented in its various embodiments as a system for harvesting the natural power of heart movement and dispatching the accumulated harvested power to power-consuming means. It is contemplated that the power consuming means can comprise, for example, and without limitation, the nominal power requirements of a pacemaker and/or a defibrillator, left ventricular assist devices, an artificial heart, of implantable sensing devices, such as for example volume and pressure sensors, lung impedance sensors, chemical sensors and the like. The convertor may be placed on the heart via a minimally invasive thoracic surgery procedure under the epicardium and over the myocardium. In an example, the longer flexible block mounted just below the atrioventricular groove, wherein the longer flexible block's ends are either mounted on the heart or mounted together, while the shorter flexible block's ends may be mounted together to form a circle, which lets moving freely without mounting. In other examples, the convertor may be placed on the diaphragms via a minimally invasive thoracic surgery procedure.

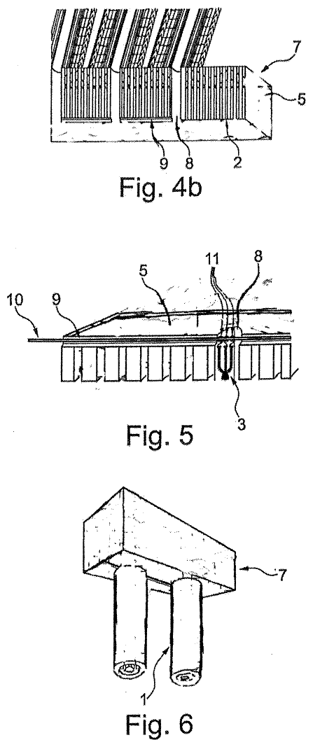

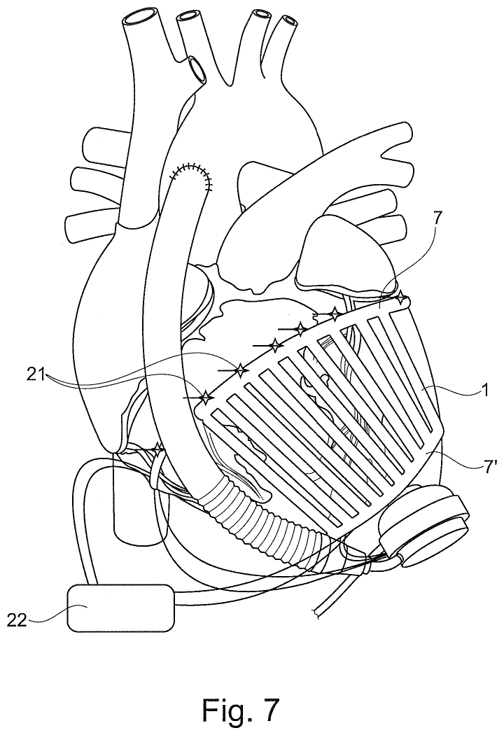

In embodiments, an convertor 100 is provided that is configured to convert the mechanical energy of the movement of the heart of a person into electrical energy. The convertor comprises a first flexible block 7 and a second flexible block 7′ being arranged to operate in two substantially parallel planes. The first and second blocks (7, 7′) may be themselves composite flexible triboelectric blocks or cylinders made of any known suitable materials, such as PTFE, PDMS, PPT, PVDF, FEP, Kapton, Nylon, PET polymers, enhanced with Al, Cu, Ag, Au, carbon or other nanofibers, and mechanically or chemically post-processed to maximize their efficiency, that allow electrical energy generation for multiple applications. The first and second flexible block (7, 7′) may be constructed through additive manufacturing (3D printing) that allows a more cost-effective solution compared to traditional production methods, such as extrusion process. The convertor may further comprise a plurality of extendable elements 1, each of them having a first end and a second end. The extendable elements are configured to act as triboelectric nanogenerators in response to a physiological force applied to the extendable elements and generate electrical power, and they are made of the same materials as the first and second blocks or cylinders, following the same principles, constructed in continuum with the same method at the same time, as depicted in FIG. 6. The respective triboelectric material layer 2, of each extendable element 1 is also produced at the same time via additive manufacturing and may be mechanically or chemically post-processed in-situ. As can be seen in FIGS. 1 and 4a, the plurality of extendable elements 1 is substantially perpendicular relative to the first and second blocks 7, 7′ and form a grid. Each first and second end of each of the plurality of the extendable elements 1 is immovably connected to the first and second flexible blocks 7, 7′ respectively, thus ensuring the structural integrity of the convertor 100.

In examples, each of the plurality of the extendable elements 1 comprises a hollow portion 6 and a triboelectric spiral structure 3 that is located within the hollow portion 6 as it can be seen in FIG. 2. Such triboelectric spiral structure 3 contributes to the production of electrical energy, taking advantage of the mechanical energy that is created from the heart's movement of a human, or from any other moving organ (lungs, diaphragm, belly, etc.). Further, each of the plurality of triboelectric spiral structures 3 may further comprise a plurality of triboelectric rods 4 that are movable within the hollow portion 6 of each elongated element 1. The plurality of rods 4 may be two, three, four, etc. rods, without departing from the teachings of the present disclosure. In the embodiment described three rods 4 are depicted, as it can be seen in FIG. 2. In examples, the first and second blocks (7, 7′) and the plurality of extendable elements 1 maybe made of the same material, characterized by high mechanical strength and increased level of elasticity, providing the capability of smooth prolongation of approximately 25-40% relative to the original size. Further, the first and second blocks (7, 7′) may be of different length. In examples, the first flexible block 7 may be of about 1,2 to 2 times longer than the second flexible block (7′). In other more specific examples, the length of the first flexible block 7 is of about 1,7 of the length of the second flexible block 7′. In other examples, the length of the first flexible block may be in the range of 20-30 cm and the length of the second flexible block may be in the range of 10-18 cm. Such difference in the length of the first and second block is desirable in order to ensure the proper mounting on the respective anatomic area of the heart and the length range of the device is desirable to fit hearts of various sizes. In detail, the device should be able to follow the heart muscle movement without any severe mechanical constraints. As it is commonly known, the heart has a substantially truncated cone shape. It has been identified after experimentation that the optimal mounting location for the first block 7 which should be the biggest, is the basis of the ventricles (a wide part of the cone) while for the second block 7′ is the apex. The first flexible block 7 is of such length and elasticity that can embrace the perimeter of the cone of the heart. In other examples, the first and second flexible blocks (7, 7′) may be of the same thickness which may range between 0.2 and 0.8 cm. In other examples, the first and second flexible blocks (7, 7′), may be equally sized, as this configuration may be needed to be mounted between the arches of the diaphragms.

In embodiments, each extendable element 1 comprises a hollow portion 6, preferably of cylindrical shape, extending longitudinally across the entire length of each extendable element, and across the entire width of both flexible blocks 7, 7′ thus creating a through hole 8, throughout the flexible blocks as it can be seen for example in FIGS. 4a and 4b. The through hole 8 is necessary for the loading of the triboelectric spiral structure inside the hollow portion 6 of the extendable element 1. The cylindrical shape is significant since it maximizes the potential of energy harvesting by providing a relatively large area of triboelectric interaction between respective material layers in a given space, during the movement of the heart or other organs of a human body. Having a plurality of extendable elements of cylindrical shape, covering a significant area of the available heart surface, provides the advantage of maximizing the triboelectric potential for this application, thus consequently harvesting the adequate amount of energy that is necessary for the proper function of an energy-consuming medical device, such as a pacemaker, as described in this disclosure. Having thus extendable elements of cylindrical shape is beneficiary compared to other known methods, such as the placement of a sheet that is just covering the heart of a human body, since it maximizes the exploitation of the energy that is produced from the heart of the human body. In examples, and as it can be seen in FIGS. 4a, 4b, the first and second flexible block (7, 7′) may comprise a first end and a second end, and a through hole 8 extending longitudinally from the first end to the second end, in each of the first and second flexible block (7, 7′).

In examples, each of the plurality of triboelectric rods 4 comprises two concentric material layers, in particular a triboelectric layer and an inner conducting material layer 12, as can be seen for example in FIG. 2. Such triboelectric rods 4 are preferably cylindrical and are interlaced so that in their plurality constitute a spiral structure 3. The structure 3 has a maximal diameter of about 25-50% of the internal diameter of the hollow portion 6 of each of the extendable elements 1. The diameter of each triboelectric element 4 may thus be of about 0.2 to 0.6 cm. Both the elongated element 1 and the triboelectric rods 4 show a significant elasticity that results to a prolongation of approximately 25-40% relative to their original size and complete elastic return to their initial condition thereof. In the same FIG. 2 it is also shown that the outermost layer is the insulating material 5.

In embodiments shown in FIG. 5, each rod 4 of the plurality of rods comprises at each end an electrical connection 11. The electrical connection 11 may be wrapped under tension to each end of each rod 4 to ensure that each rod is kept steady during operation of the convertor 100. Further, the conducting material layer 12 of each rod 4 of the plurality of rods 3 may be removably connected to a metal wire 10 that extends through the through hole 9 of each flexible block 7, 7′. In that way, the rods 4 are securely and firmly connected to the various components of convertor 100. The wire 10 may have a thickness of about 0.3 cm and 0.5 cm and may be made of any suitable material such as but not limited to steel or nickel alloys. In a preferred embodiment, three rods 4 comprising the triboelectric layer and the conducting layer 12, form a unified triboelectric spiral structure 3. When a triboelectric spiral structure 3 is formed, it is inserted into the corresponding elongated element 1 through relevant holes 8, which may then be sealed with silicone.

In embodiments, additional metallic wires of the same material and thickness as wire 10 are mounted on the end of the flexible blocks, into the conducting material component of the flexible block, and close the electrical circuit among the triboelectric layer 2 and the triboelectric rods 4.

In embodiments, when convertor 100 is in its fully assembled status, it is finally coated to all of its external surfaces with a mixture 5 comprising an electrically insulating material and an anti-fibrotic solute, to avoid any reactive tissue fibrosis, as it can be seen in for example in FIGS. 2, 3, 4a, 4b, 5. The coating process may be performed through electro-spraying or any other suitable method. The electrical insulating material may be selected from any suitable polymer material, such as but not limited to polytetrafluoroethylene (PTFE) or rubber. The anti-fibrotic solute may comprise magnesium salt and dimethoxyphenyl-propanol-aminobenzoic acid. Such materials provide anti-fibrotic properties when they are embedded in coatings of, for example, devices that are implanted in a human body, mitigating the risk of any undesired reaction from the human body due to the implant of the device.

The convertor 100 according to the present disclosure may be used during a surgical operation by being placed through a minimally invasive procedure, under the epicardium, and over the myocardium, around the heart of the human body, the second flexible block 7′ may be fixed to the myocardium of the base of the ventricles with non-absorbable sutures, just under the atrioventricular groove, while other sutures connect to each end of the second flexible block 7′ such that the second flexible block remains constant along the transverse axis of the heart. Further, the first flexible block 7 which surrounds the top of the heart of a human body, is not riveted as the second flexible block; only its ends are connected to each other with sutures so that it follows smoothly the heart movement. The convertor 100 may be placed across the diaphragms arches with both flexible blocks mounted on the diaphragm. Moreover, from each end of the second flexible block 7′, electrical power cables are coming out and are connected to a voltage stabilizer-electrical management system and an accumulator. The voltage stabilizer and the accumulator may be located in a casing preferably made of silicon, in the vicinity of the heart of the human body. The surgical operation may be combined with a simultaneous implantation of another medical device, for example a mechanical pump, at the top of the left ventricle of the heart, in the chest, which is connected to the convertor and the rest of the system (voltage stabilizer and accumulator) to the casing.

In embodiments, the convertor may also comprise an additional electrical device capable of transmitting electromagnetic waves, thus providing the advantage of wireless transmission of data measurements of the electrical voltage and current that goes through the convertor. The wireless transmission can also provide useful information on the functioning of the heart or other organs. Such transmission, which can also be quasi-continuous, can only be achieved if a power generation mechanism is permanently implanted in the human body.

In embodiments, the convertor may also comprise an additional external layer of nanowires capable of delivering electrical stimulation directly on the myocardium.

FIG. 7 shows an embodiment of the present disclosure where the convertor is mounted on a heart muscle. The upper flexible block 7 is mounted to the heart through stitches 21. The energy management system 22 is electrically connected to the upper—7 and the lower—7′ solid blocks and comprises known to the art features which receive and store electric energy.

It should be noted that the above embodiments are only for illustrating and not limiting the technical solutions of the present invention. Although the present invention has been described in detail with reference to the above embodiments, those skilled in the art should understand that any modifications or equivalent substitutions of the present invention are intended to be included within the scope of the appended claims.

Although the present disclosure herein has been described with reference to particular embodiments, it is to be understood that these embodiments are merely illustrative of the principles and applications of the present disclosure.

### REFERENCE SIGNS

1: extendable elements
2: triboelectric layer
3: triboelectric spiral structure
4: triboelectric rods (elongated elements)
5: insulating material
6: hollow portion of extendable element
7, 7: flexible blocks
8: hole of extendable element
9: through hole of each flexible block
10: wires
11: electrical connections
12, 12′: conducting material layers.
21: stitches
22: energy management system

1. A convertor of mechanical energy for converting the mechanical energy of the movement of the heart muscle into electrical energy comprising:
a first flexible block
a second flexible block
wherein a first and a second end of a plurality of extendable elements are immovably connected on the first and on the second flexible blocks, wherein each of the plurality of extendable elements comprises a layer of triboelectric material and a hollow portion, wherein each of the plurality of the extendable elements comprises a triboelectric spiral structure that is mounted within the hollow portion, wherein the triboelectric spiral structure comprises a plurality of twisted elongated elements that are movable within the hollow portion of the extendable elements.

2. The convertor of claim 1, wherein each of the plurality of the elongated elements has the shape of a rod and wherein each of the elongated elements comprises a triboelectric material layer and a conducting material layer.

3. The convertor of claim 2, wherein the triboelectric layer of the extendable elements and the triboelectric layer of the elongated elements come in direct contact by movement.

4. The convertor of claim 1, wherein the plurality of twisted elongated elements consists of three rods.

5. The convertor of claim 1, wherein the length of the first flexible block is about 1.2 to 2 times longer than the second flexible block.

6. The convertor of claim 1, wherein the length of the first flexible block is 1.7 times longer than the second block.

7. The convertor of claim 1, wherein each of the plurality of extendable elements has a cylindrical shape.

8. The convertor of claim 1, wherein each of the plurality of extendable elements is made of a flexible material having an original length, said flexible material being capable of elongating of up to 40% compared to its original length and configured to return to its original length, upon ending of its elongation.

9. The convertor of claim 1, further comprising a coating comprising a mixture of an electrical insulating material and an anti-fibrotic solute.

10. The convertor according to claim 9, wherein the anti-fibrotic solute comprises magnesium salt and dimethoxyphenyl-propanol-aminobenzoic acid.

11. The convertor of claim 1, wherein the first and second flexible blocks further comprising a first end and a second end and a through hole extending longitudinally from the first end to the second end of each of the first and second flexible block.

12. The convertor of claim 11, wherein each elongated element from the plurality of elongated elements further comprises at least one electrical connection wherein the electrical connection is wrapped under tension to the elongated element.

13. The convertor of claim 12, wherein the conducting layer of each elongated element is coupled with a metal wire, said wire extending through the through hole of each flexible block.

14. Use of the convertor of claim 1 for converting the mechanical energy of the movement of an organ of the human body into electrical energy.

15. Use of the convertor according to claim 14 with a system comprising a voltage stabilizer and an accumulator, configured to produce electrical power derived by the contraction and the expansion of the heart muscle of a human body.
